(19) United States
(12) Patent Application Publication
(10) Pub. No.: US 2003/0070485 A1
(43) Pub. Date: Apr. 17, 2003

(54) Method for setting tone controls during a hearing test

(76) Inventors: Benny B. Johansen, Sunnyvale, CA (US); Ephram Cohen, San Francisco, CA (US); Sunil Puria, Mountain View, CA (US)

Correspondence Address: HAYNES BEFFEL & WOLFELD LLP, P O BOX 366, HALF MOON BAY, CA, 94019, US

(21) Appl. No.: 09/975,581

(22) Filed: Oct. 11, 2001

(51) Int. Cl. A61B 5/00; A61B 5/12

(52) U.S. Cl. 73/585; 600/559

(57) ABSTRACT

A method of testing the hearing of a user utilizing a computer system that includes a computer and a speaker. The computer can output an electrical signal to the speaker and the speaker is operable to convert the electrical signal into a stimulus. The computer system has a tone control that controls the spectral components of the electrical signal. The method includes: downloading a computer program from a server to the computer; executing the computer program on the computer, the execution of the computer program setting the tone control; generating a stimulus; and receiving an input from the user that indicates whether the user heard the stimulus.

This patent application discloses subject matter that is related to the subject matter disclosed in U.S. patent application Ser. No. ______ entitled “Method For Setting Volume and/or Balance Controls During a Hearing Test,” Ser. No. ______ entitled “Method For Muting and/or Un-Muting of Audio Sources During a Hearing Test,” and Ser. No. ______ entitled “Method and System For Generating Audio Streams During a Hearing Test,” filed on even date herein. Each of the above patent applications is hereby incorporated by reference.

### FIELD OF THE INVENTION

The present invention generally relates to audiology and the presentation of audio signals for assessing a person's hearing using a personal computer. More specifically, the present invention relates to a system and a method for setting tone controls during a hearing test.

### BACKGROUND

Audiometric equipment exists for testing hearing. However, such equipment is expensive and is generally available only in hearing clinics. Many people are reluctant to visit hearing clinics and take a hearing test for a variety of reasons. Such reasons may include the cost of a hearing test, the time and inconvenience involved in scheduling of an appointment, waiting for and undergoing a hearing test, and privacy concerns. As a result, as many as 80 percent of the people who suffer from hearing loss in the United States may have not had their hearing tested.

Currently, a number of companies provide hearing tests over the Internet. For example, a user may have his/her hearing tested by accessing one of the following URLs: www.didyouhearme.com, www.handtronix.com, www.onlinehearing.com, www.audiainc.com, www.NigelWorks.com, www.audiologvawareness.com or www.freehearingtest.com.

For example, when a user accesses www.didyouhearme.com's hearing test, the user's computer system outputs a 500 Hz tone to a speaker, such as a powered or un-powered speaker or headphone that may include piezo electric transducers, which is coupled to the computer system. Next, the website instructs the user to decrease the volume on the user's computer until the user can no longer hear the 500 Hz tone.

Next, the website generates a 4000 Hz tone. According to the website, if the user cannot hear the 4000 Hz tone, the user may have a hearing impairment. Next, the website generates a number of tones at different amplitudes at the following frequencies: 250, 500, 750, 1000, 1500, 2000, 3000, and 4000 Hz. When the user hears a particular frequency, the user presses an acknowledge button on the screen with the computer's mouse. Based upon the user's acknowledgements, the website generates an audiogram. An audiogram is a chart plotting a user's hearing threshold level in dB HL as a function of frequency. Using the audiogram, the user can determine if the user has a hearing impairment.

Modern computer systems often have one or more tone controls that adjust the spectral characteristics of sounds output by the computer's speakers. For example, many Windows operating systems include a window entitled “Advanced Controls for Volume Control.” A diagram of a portion of this window is shown in FIG. 1. Referring to FIG. 1, the window 100 includes two tone controls. The first tone control 105, a horizontal slider bar labeled “Bass,” controls the amplitude of the low-frequency components of sounds output by the computer's speakers. The second tone control 110, another horizontal slider bar labeled “Treble,” controls the amplitude of high-frequency components of sounds output by the computer's speakers.

In addition to the above tone controls 105 and 110, some computer systems utilize additional tone controls such as graphic equalizers. As is well known, a window displaying a graphic equalizer (not shown) allows a user to adjust the amplitude of different frequency bands of sounds that are output by the computer's speakers.

Unfortunately, the accuracy of the above hearing test can be significantly affected by tone control settings. For example, if tone controls are adjusted so that a 500 Hz frequency is much louder than a 4000 Hz frequency, then a user may mistakenly believe that he/she has a hearing impairment. On the other hand, if the tone controls are adjusted so that a 500 Hz frequency is much softer than a 4000 Hz frequency, then a user may mistakenly believe that he/she has normal hearing when, in fact, the user has a hearing impairment.

Thus, a need exists for a hearing test that avoids such inaccuracies.

### SUMMARY OF THE INVENTION

One embodiment of the invention is a method of testing the hearing of a user utilizing a computer system that includes a computer and a speaker. The computer can output an electrical signal to the speaker and the speaker is operable to convert the electrical signal into a stimulus. The computer system has a tone control that controls the spectral characteristics of the electrical signal. The method includes: downloading a computer program from a server to the computer; executing the computer program on the computer, the execution of the computer program setting the tone control; generating a stimulus; and receiving an input from the user that indicates that the user heard the stimulus. In some embodiments the method also includes: sending data to the server; qualifying the hearing of the user; and sending other data to the computer.

Another embodiment of the invention is another method of testing the hearing of a user utilizing the above computer system. This method includes: downloading a computer program from a server to the computer; executing the computer program on the computer, the execution of the computer program storing the value of the tone control and setting the tone control; generating a stimulus; receiving an input from the user that indicates whether the user heard the stimulus; and resetting the tone control to the stored value. In some embodiments the method also includes: sending data to the server; qualifying the hearing of the user; and sending other data to the computer.

Still other embodiments of the invention include program storage devices that contain computer readable instructions that, when executed, perform portions of the above methods.

### BRIEF DESCRIPTION OF THE FIGURES

FIG. 1 presents an “Advanced Controls for Volume Control” applet window.

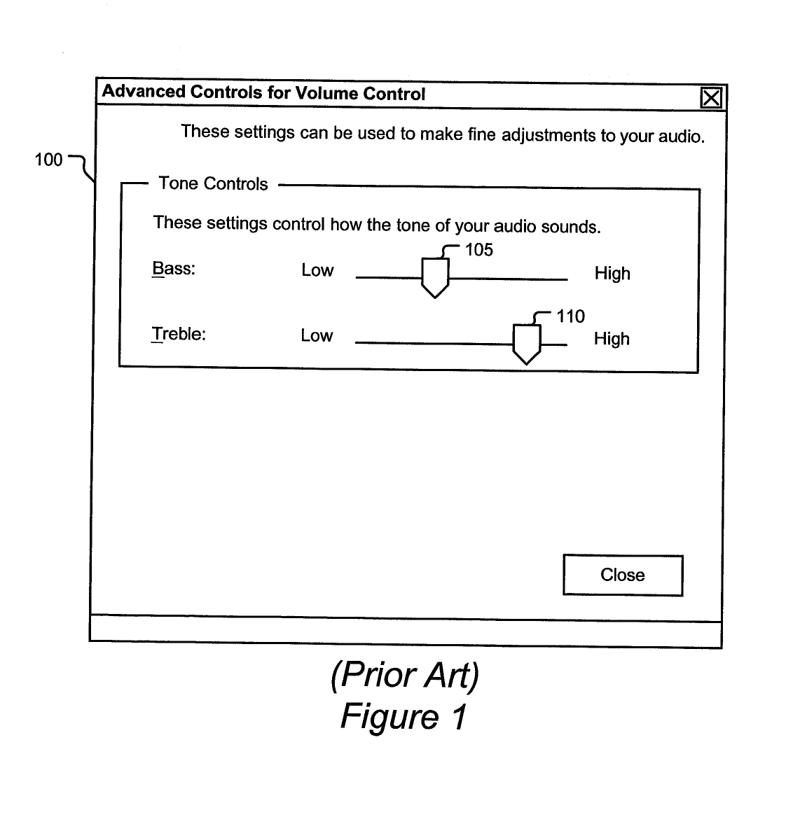

FIG. 2 presents a method of testing the hearing of a user that includes setting a tone control.

FIG. 3 presents another method of testing the hearing of a user that includes first setting a tone control and then resetting it later.

### DESCRIPTION OF THE PREFERRED EMBODIMENTS

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

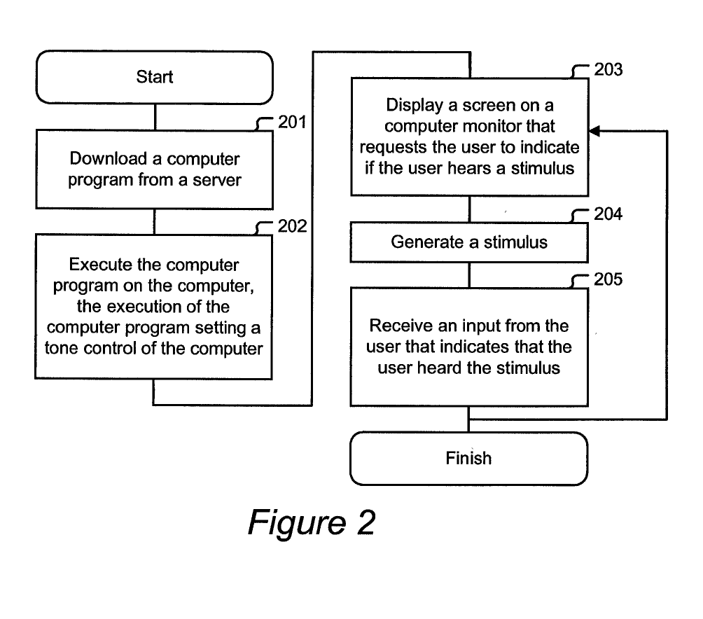

One embodiment of the invention, a method of testing the hearing of a user utilizing a computer system, is shown in FIG. 2. The method can be performed on a conventional computer system, such as a desktop computer system, a laptop computer system, or a handheld computer system. Other devices that include a microprocessor, such as a telephone, a mobile phone, a personal display assistant, an MP3 player, a radio, or a television, can also perform the method. Hearing Tests that Set Tone Controls

Referring to Block 201 of FIG. 2, a user that desires to take a hearing test first downloads a computer program, such as a stand-alone executable program, a Java applet, an Active X control, or a Netscape plugin, from another computer, such as a server, to his/her computer. In one embodiment of the invention, the computer program is transferred via the Internet. In another embodiment of the invention, the computer program is transferred via an email. As is well known, computer programs may be attached to emails that can be easily distributed over the Internet, virtual private networks, local area networks and/or wide area networks. In still other embodiments, the computer program could be transferred to the user via the United States postal service or other postal service.

Next, referring to Block 202 of FIG. 2, the user executes the computer program on the user's computer. The execution of the computer program sets a tone control, such as the above-described “Bass” tone control 105, to a predefined tone level such as 50%. In some embodiments of the invention, the computer program would also set the “Treble” tone control 110 to 50%. In other embodiments of the invention, the computer program would also set one or more tone controls in a graphic equalizer.

Next, referring to Block 203 of FIG. 2, the computer program displays a screen on the computer monitor that requests the user to indicate if the user hears a stimulus. Then, referring to Block 204 of FIG. 2, the computer program outputs a stimulus. If the user hears the stimulus, then the user inputs information, such as via a mouse click or a keyboard entry, into the computer that indicates whether the user hears the stimulus. Referring to Block 205 of FIG. 2, the computer program then receives the input from the user that indicates that the user heard the stimulus.

As shown in FIG. 2, by repeating Blocks 203 through 205 with stimuli of different amplitudes and frequencies, data sufficient to quantify the hearing of the user can be derived using conventional methods. In some embodiments of the invention, the computer program qualifies the hearing. In other embodiments, the computer program transfers data to a server and the server qualifies the hearing and then sends data back to the computer program. After the hearing of the user is quantified, some embodiments of the invention present an audiogram, text information, and/or graphical information to the user.

By setting one or more tone controls to predefined levels, the accuracy of the hearing test may be increased. For example, if the “Bass” tone control 105 is initially set to a very low level, then, unless the tone control 105 is reset, the computer program may not be able to generate a low-frequency stimulus that can be heard by the user. On the other hand, if the “Bass” tone control 105 is initially set to a very high level, then, unless the tone control 105 is reset, the computer program may generate a “clipped” electrical signal that results in a distorted stimulus. In addition, by setting the tone controls 105 and 110 to predefined levels, the relative amplitudes of high-frequency electrical signals to low-frequency electrical signals can be accurately controlled so that accurate hearing tests may be performed.

Other Embodiments of the Invention

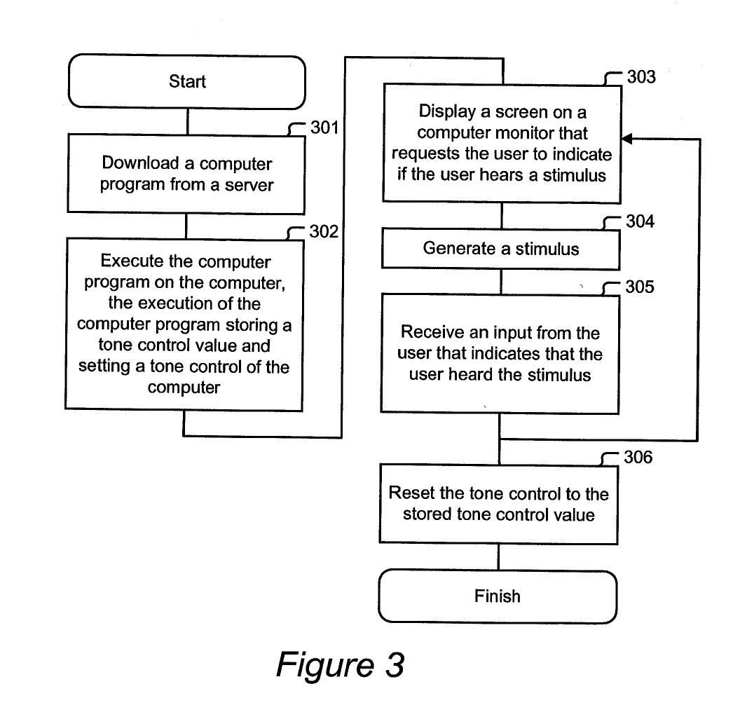

If the user had previously set one or more tone controls, then the above method would “overwrite” the previously set tone control value(s). Thus, in some embodiments of the invention, such as shown in FIG. 3, the computer program would store one or more previously set tone control values before setting the tone controls. Then, after the conclusion of the hearing test, the computer program would reset the tone controls to the previously set values.

Conclusion

The foregoing descriptions of embodiments of the present invention have been presented for purposes of illustration and description only. They are not intended to be exhaustive or to limit the present invention to the forms disclosed. Accordingly, many modifications and variations will be apparent to practitioners skilled in the art. For example, program storage devices, such as hard disks, floppy disks, random access memories (RAM), read only memories (ROM), programmable read only memories (PROM), compact disks (CD), and digital versatile disks that contain computer readable instructions that perform portions of the above methods, are intended to be included in the present invention. Additionally, the above disclosure is not intended to limit the present invention. The scope of the present invention is defined by the appended claims.

It is claimed:

1. A method of testing the hearing of a user utilizing a computer system, the computer system including a computer and a speaker, the computer operable to output an electrical signal to the speaker, the speaker operable to convert the electrical signal into a stimulus, the computer system having a tone control that controls the spectral characteristics of the electrical signal, the method comprising:
a) downloading a computer program from a server to the computer;
b) executing the computer program on the computer, the execution of the computer program setting the tone control;
c) generating a stimulus; and
d) receiving an input from the user that indicates whether the user heard the stimulus.

2. The method of claim 1, wherein the act of downloading the computer program includes transferring the computer program from the server to the computer via the Internet.

3. The method of claim 1, wherein the act of downloading the computer program includes transferring the computer program from the server to the computer via an email.

4. The method of claim 1, wherein the act of executing the computer program includes setting a tone control that controls the amplitude of low-frequency components in the electrical signal.

5. The method of claim 1, wherein the act of executing the computer program includes setting a tone control that controls the amplitude of high-frequency components in the electrical signal.

6. The method of claim 1, wherein the act of executing the computer program includes setting a tone control within a graphic equalizer.

7. The method of claim 1, wherein the act of executing the computer program includes setting a first tone control that controls the amplitude of low-frequency components in the electrical signal and setting a second tone control that controls the amplitude of high-frequency components in the electrical signal.

8. The method of claim 1, further including:
a) sending first data to the server;
b) qualifying the hearing of the user; and
c) sending second data to the computer.

9. A method of testing the hearing of a user utilizing a computer system, the computer system including a computer and a speaker, the computer operable to output an electrical signal to the speaker, the speaker operable to convert the electrical signal into a stimulus, the computer system having a tone control that controls the spectral characteristics of the electrical signal, the method comprising:
a) downloading a computer program from a server to the computer;
b) executing the computer program on the computer, the execution of the computer program storing the value of the tone control and setting the tone control;
c) generating a stimulus;
d) receiving an input from the user that indicates whether the user heard the stimulus; and
e) resetting the tone control to the stored value.

10. The method of claim 9, wherein the act of downloading the computer program includes transferring the computer program from the server to the computer via the Internet.

11. The method of claim 9, wherein the act of downloading the computer program includes transferring the computer program from the server to the computer via an email.

12. The method of claim 9, wherein the act of executing the computer program includes setting a tone control that controls the amplitude of low-frequency components in the electrical signal.

13. The method of claim 9, wherein the act of executing the computer program includes setting a tone control that controls the amplitude of high-frequency components in the electrical signal.

14. The method of claim 9, wherein the act of executing the computer program includes setting a tone control within a graphic equalizer.

15. The method of claim 9, wherein the act of executing the computer program includes setting a first tone control that controls the amplitude of low-frequency components in the electrical signal and setting a second tone control that controls the amplitude of high-frequency components in the electrical signal.

16. The method of claim 9, further including:
a) sending first data to the server;
b) qualifying the hearing of the user; and
c) sending second data to the computer.

17. A program storage device that contains computer readable instructions that, when executed by a computer system having a tone control, tests the hearing of a user by:
a) setting the tone control of the computer;
b) generating a stimulus; and
c) receiving an input from the user that indicates whether the user heard the stimulus.

18. The program storage device of claim 17, wherein the act of setting the tone control includes setting a tone control that controls the amplitude of low-frequency components of an electrical signal.

19. The program storage device of claim 17, wherein the act of setting the tone control includes setting a tone control that controls the amplitude of high-frequency components of an electrical signal.

20. The program storage device of claim 17, wherein the act of setting the tone control includes setting a first tone control that controls the amplitude of low-frequency components of an electrical signal and a second tone control that controls the amplitude of high-frequency electrical components of the electrical signal.

21. A program storage device that contains computer readable instructions that, when executed by a computer system having a tone control, tests the hearing of a user by:
a) storing the value of the tone control;
b) setting the tone control;
c) generating a stimulus;
d) receiving an input from the user that indicates whether the user heard the stimulus; and
e) resetting the tone control to the stored value.

22. The program storage device of claim 21, wherein the act of setting the tone control includes setting a tone control that controls the amplitude of low-frequency components of an electrical signal.

23. The program storage device of claim 21, wherein the act of setting the tone control includes setting a tone control that controls the amplitude of high-frequency components of an electrical signal.

24. The program storage device of claim 21, wherein the act of setting the tone control includes setting a first tone control that controls the amplitude of low-frequency components of an electrical signal and a second tone control that controls the amplitude of high-frequency electrical components of the electrical signal.
